(19) United States
(12) Patent Application Publication
(10) Pub. No.: US 2020/0194771 A1
(43) Pub. Date: Jun. 18, 2020

(54) MEDICAL ELECTRODES HAVING ENHANCED CHARGE CAPACITIES, AND METHODS OF MANUFACTURING

(71) Applicant: NeuroTronik IP Holding (Jersey) Limited, St. Helier (JE)

(72) Inventor: Scott Purcell, Raleigh, NC (US)

(21) Appl. No.: 16/712,931

(22) Filed: Dec. 12, 2019

Related U.S. Application Data
(60) Provisional application No. 62/778,352, filed on Dec. 12, 2018.

(51) Int. Cl. H01M 4/04; H01L 51/00; A61N 1/04

(52) U.S. Cl. CPC H01M 4/0404; A61N 1/0456; H01L 51/0037

(57) ABSTRACT

A method of manufacturing a high charge capacity electrode for delivering electrical energy to target tissue of a patient comprises performing a laser process on a conductive material to increase its surface topography, and then coating the conductive material with a high specific area coating.

This application claims the benefit of U.S. Provisional Application No. 62/772,352, filed Dec. 12, 2019.

### TECHNICAL FIELD OF THE INVENTION

The present application generally relates electrodes for delivering energy or stimulus to tissue or structure of the body. More specifically, the application relates to electrode manufacturing processes.

### BACKGROUND

Co-pending U.S. application Ser. No. 13/547,031 entitled System and Method for Acute Neuromodulation, filed Jul. 11, 2012 (Attorney Docket: IAC-1260; the “'031 application”), filed by an entity engaged in research with the owner of the present application, describes a system which may be used for hemodynamic control in the acute hospital care setting, by transvascularly directing therapeutic stimulus to parasympathetic nerves and/or sympathetic cardiac nerves using electrodes positioned in the superior vena cava (SVC). In disclosed embodiments, delivery of the parasympathetic and sympathetic therapy decreases the patient's heart rate (through the delivery of therapy to the parasympathetic nerves) and elevates or maintains the blood pressure (through the delivery of therapy to the cardiac sympathetic nerves) of the patient in treatment of heart failure.

Co-pending U.S. application Ser. No. 14/642,699 (the '699), filed Mar. 9, 2015 and U.S. Ser. No. 14/801,560 (the '560), filed Jul. 16, 2015, each incorporated by reference, describe transvascularly directing therapeutic stimulus to parasympathetic and/or sympathetic cardiac nerves using electrodes positioned in the SVC, right brachiocephalic vein, and/or left brachiocephalic vein and/or other sites. As with the system disclosed in the '031, the methods disclosed in these applications can decrease the patient's heart rate (through the delivery of therapy to the parasympathetic nerves) and elevate or maintain the blood pressure (through the delivery of therapy to the cardiac sympathetic nerves) of the patient in treatment of heart failure.

The '699 and '560 applications describe one form of catheter device that may be used to perform transvascular neuromodulation. In particular, these applications shows a support or electrode carrying member 10 of the type shown in FIG. 1 on the distal part of a catheter member 4. The electrode carrying member 10 includes a plurality of struts 12. One or more of the struts carries one or a plurality of electrodes 17. The electrode carrying member 10 is designed to bias such electrodes into contact with the vessel wall. The material forming the struts 12 may have a shape set or shape memory that aids in biasing the circumferentially-outward facing surfaces (and thus the electrodes) against the vessel wall. The applications describe that the electrodes 17 may be mounted to or formed onto a substrate 15 that is itself mounted onto a strut or a plurality of strut. It is also disclosed that the struts and electrodes may use flex circuit or printed circuit elements.

Co-pending and commonly owned U.S. application Ser. No. ______ (Attorney Ref: NTK2-2010), filed Dec. 12, 2019 and incorporated herein by reference, describes electrode support assemblies in which flexible circuits (having electrodes and/or other components on them) may be mounted to an electrode support. Referring to FIGS. 2A and 2B, the method makes use of a heat staking process to fix a flexible circuit 100 to a strut 102, such as a shape memory strut formed of nitinol or alternative material. The flexible circuit includes the electrodes 103. In general, to carry out the method, the shank of the rivet is passed through a hole 110 in the flexible circuit and an aligned hole 112 in the strut as indicated in FIG. 2A. The rivet is then subjected to a heating process that causes the end of the shank to expand radially and compress longitudinally, forming a secondary head on the opposite end of the shank from the first head. The flexible circuit and strut are thus fixed to one another, captured between the primary head and secondary head of the rivet.

Concepts described in the present application may be used to create an electrode surface, which may on a flex circuit, capable of achieving the current densities needed to carry out the therapy performed in the referenced applications, and for the durations at which therapy could be applied in the acute setting (e.g. up to 96 hours).

### BRIEF DESCRIPTION OF THE DRAWINGS

FIG. 1 shows an electrode carrying member of the type shown in the '699 and '560 applications, with electrodes carried thereon.

FIG. 2A shows components of an electrode carrying member prior to assembly, and illustrates a method of assembling the flexible circuit to a strut on the array. FIG. 2B shows the electrode carrying member following assembly with the flexible circuit.

FIG. 3A is a flow diagram showing a sequence of steps in a first manufacturing method for enhancing the charge capacity of electrodes.

FIG. 3B is a flow diagram showing a sequence of steps in a second manufacturing method for enhancing the charge capacity of electrodes.

### DETAILED DESCRIPTION

This application describes processes that may be used to create an electrode surface capable of achieving the current densities needed to carry out the therapy performed in the referenced applications, and for the durations at which therapy could be applied in the acute setting (e.g. up to 96 hours). These processes make use of a laser skiving process or other suitable process to enhance the topography of the electrodes, thus making the electrodes cable of achieving higher charge capacities, and they additionally apply a high specific surface area materials (e.g. IrOx or PEDOT) to the electrode surface to further increase the effective surface area and thus the storage capacity of the electrode surface.

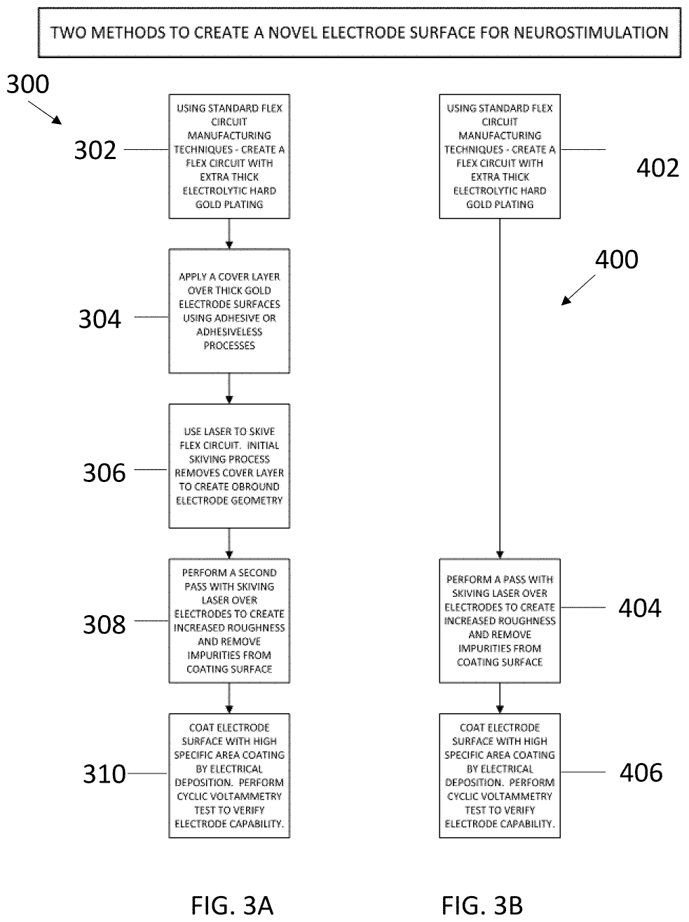

In accordance with a first example of an electrode manufacturing method 300 illustrated in FIG. 3A, a flexible circuit is created using flex circuit manufacturing techniques known to those skilled in the art. The flex circuit is plated with thick, electrolytic hard gold plating. Step 302. A cover layer is applied over the gold electrode surface using an adhesive or adhesiveless process. Step 304. In step 306, a laser is used to skive the flex circuit. This initial skiving step removes portions of the cover layer to expose electrodes on the flex circuit. In preferred examples, the electrodes have an obround geometry, but in other examples electrodes of any shape may be formed. A second laser skiving step is performed in step 308. In this second skiving step, a skiving laser is passed over the flex circuit to create increased roughness in the gold electrode material and to remove impurities from the coating surface. Finally, in step 310, the gold electrode surface is coated with a high specific area coating, preferably using electrical deposition. A cyclic voltammetry test may be performed on the electrodes to verify their capability.

A second example of a manufacturing process 400 is shown in FIG. 3B. In this embodiment, a flexible circuit is created using flex circuit manufacturing techniques known to those skilled in the art. The flex circuit is plated with thick, electrolytic hard gold plating. Step 402. A laser skiving step is performed in step 404. In this skiving step, a skiving laser is passed over the flex circuit to create increased roughness in the gold electrode material and to remove impurities. Finally, in step 406, the gold electrode surface is coated with a high specific area coating, preferably using electrical deposition. A cyclic voltammetry test may be performed on the electrodes to verify their capability.

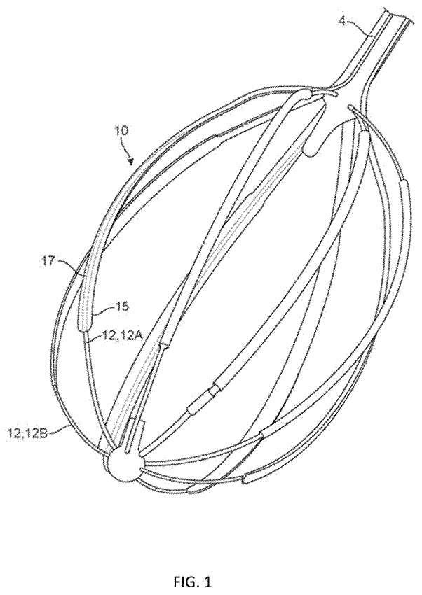

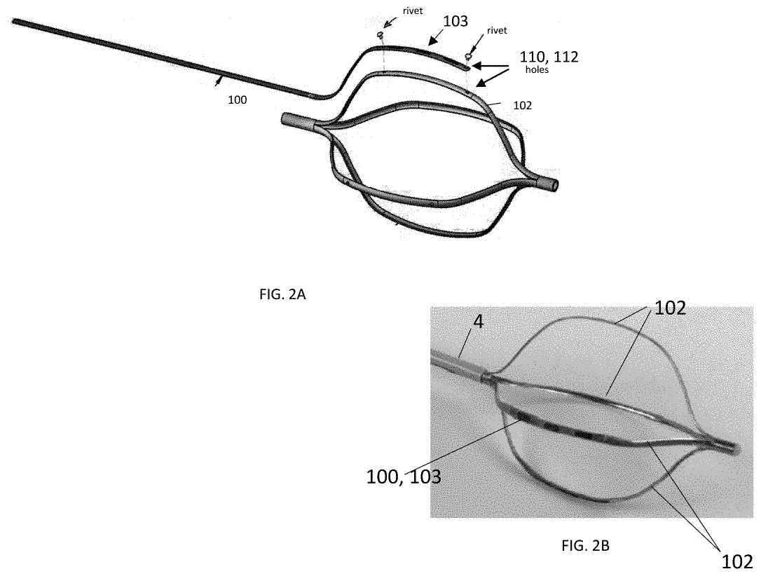

The processes described above result in creation of a flexible circuit having one or more electrodes, each of which possesses an electrode surface particularly suitable for transvascular stimulation of nerve targets in therapies such as those described in the referenced applications, which require high charge densities over extended time periods (e.g. up to 96 hours). The flexible circuits may be mounted to electrode carrying members such as the strut arrangements described above with respect to FIGS. 1-2B, or alternative electrode carrying members for intravascular or extravascular use.

All applications and patents referred to herein, including for purposes of priority, and incorporated herein by reference.

What is claimed is:

1. A method of manufacturing an electrode for use in delivering electrical energy to target tissue of a patient, the method comprising:
providing a conductive material;
performing a laser process on the conductive material to increase its surface topography; and
coating the conductive material with a high specific area coating.

2. The method according to claim 1, wherein the method includes providing a flexible circuit having the conductive material thereon.

3. The method according to claim 1, wherein the conductive material is electrolytic hard gold plating on a flexible circuit.

4. The method of claim 1, wherein the high specific area coating is IrOx.

5. The method of claim 1, wherein the high specific area coating is PEDOT.

6. The method of claim 1, further including the step of, before performing the laser process, applying a cover layer over the conductive material.

7. The method of claim 6, wherein the cover layer is applied using an adhesiveless process.

8. The method of claim 6, wherein the cover layer is applied using an adhesive process.

9. The method of claim 6, wherein the laser process is a second laser process, and wherein the method further includes the step of performing an initial laser skiving step to remove a portion of the cover layer, to expose an electrode of a desired shape and size.

10. The method of claim 9, wherein the second laser process additional removes impurities from the coating.

11. The method of claim 1, wherein the laser process is a laser skiving process.

12. The method of claim 2, further including the step of mounting the flexible circuit to an electrode carrying member proportioned for positioning in venous vasculature superior to the heart of a human adult for delivery, using the electrodes, of the electrical energy to nerve targets outside the brachiocephalic vein.

13. The method of claim 12, wherein the electrode carrying member is proportioned for positioning in brachiocephalic vein of a human adult to permit placement of the electrodes in contact with a posterior surface of said brachiocephalic vein.
